(19) United States
(12) Patent Application Publication
(10) Pub. No.: US 2001/0042550 A1
(43) Pub. Date: Nov. 22, 2001

(54) Device to avoid chafing of the feet when wearing new shoes

(76) Inventors: Francisco Osuna Fernandez, Alicante (ES); Luis Osuna Fernandez, Alicante (ES)

Correspondence Address: Evenson, McKeown, Edwards & Lenahan, P.L.L.C., Suite 700, 1200 G Street, N.W., Washington, DC, 20005, US

(21) Appl. No.: 09/813,953

(22) Filed: Mar. 22, 2001

(30) Foreign Application Priority Data
May 19, 2000 (ES) 200001334/1

(51) Int. Cl. A61F 5/37

(52) U.S. Cl. 128/889; 128/846

(57) ABSTRACT

A device avoids chafing on the feet due to the use of new shoes. This device is formed by a complete body of flexile material such as plastic that has a generally prismatic shape open on one of the lateral sides and on the upper base. On the lower face and on the adjacent at the rear, openings or holes ventilate the rear part of the foot when the device is placed inside the rear part of the shoe.

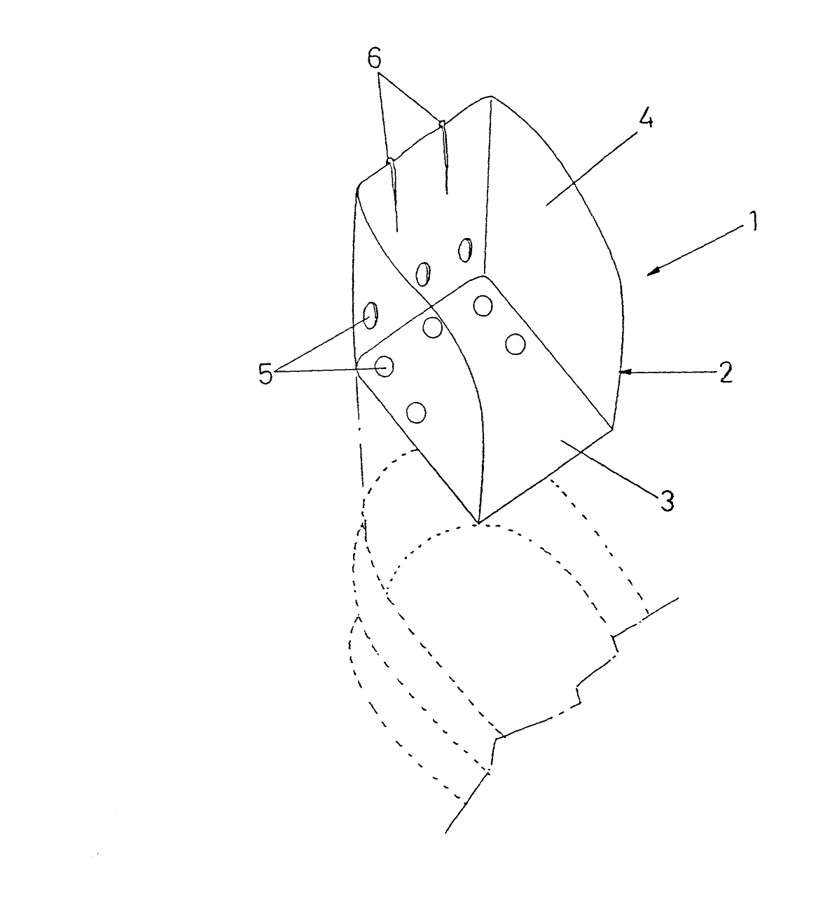

### BACKGROUND OF THE INVENTION

This application claims the priority of Spanish Application 200001334/1, filed May 19, 2000, the disclosures of which are expressly incorporated by reference herein.

An object of the present invention is to provide a specially designed device to avoid chafing of the feet when wearing shoes, preferably new shoes.

The greatest problem found when using new footwear or shoes is that chafing of the feet generally occurs, principally in the area of the heel.

This chafing is very uncomfortable and sometimes does not permit the shoes to be worn for some days.

It is therefore evident that this problem must be taken into account because it is suffered directly by the user.

### SUMMARY OF THE INVENTION

The device of the present invention avoids the cause of chafing on the heel of the foot when in contact with the rear internal part of the shoe.

Also, this device has perforations in appropriate zones to allow that zone of the foot to be ventilated and the corresponding sweating avoided.

Another characteristic of the device of the invention consists of its having a lateral surface with slight folds that allow it to adapt perfectly to the inner rear zone of the shoe.

These folds are not uncomfortable for the user.

The production of the device is simple and very economical.

This device is an item that can be easily fitted onto the rear part of the shoe, or to footwear in general, since its design allows it to adapt to the shape of any shoe.

The constituting materials of which it is made are preferably paper or similar and as well as plastic products produced from petroleum.

The positioning and placement of the device is simple, placing it inside or outside the sock, stocking, etc. and it can be used in footwear for men, women and children.

According to the invention, the device is formed by a flexible laminar plastic body with a prismatic shape in which both the front side part and the upper base are open.

Furthermore, the rear face has small folds in a vertical direction to allow and facilitate the placing and adaptation of the device to the rear end of the shoe.

### BRIEF DESCRIPTION OF THE DRAWING

Other objects, advantages and novel features of the present invention will become apparent from the following detailed description of the invention when considered in conjunction with the accompanying drawings wherein:

The sole FIGURE is a perspective view of the device of the present invention positioned with respect to the rear internal end of a shoe shown in dotted lines.

### DETAILED DESCRIPTION OF THE DRAWING

The device 1 is formed by a laminar element 2, flexible and preferably molded in plastic.

The device therefore describes a body that adapts to the rear internal part of the shoe.

This body has a lower flat part 3 that is prolonged laterally into a lateral surface 4 of a piece with the flat part.

In the figure of the body is shown with a generally prismatic shape open on one lateral side and on the upper base.

The flat part 3 has a series of holes or openings 5 as does the rear part or face which also has these holes 5 in the same or different positions.

In order for the laminar body or element to adapt to the inside of the shoe, this element has small vertical folds 6.

The holes 5 are specially laid out to impede sweating on this part of the foot.

The foregoing disclosure has been set forth merely to illustrate the invention and is not intended to be limiting. Since modifications of the disclosed embodiments incorporating the spirit and substance of the invention may occur to persons skilled in the art, the invention should be construed to include everything within the scope of the appended claims and equivalents thereof.

1. Device to avoid foot chafing when using new shoes; comprising a complete body of flexible material that has a generally prismatic shape open on one of plural sides and on an upper base; and on a lower side and on a side adjacent the rear, openings or holes for ventilation of a rear part of a wearer's foot when the device is placed inside a rar part of the a shoe.

2. Device according to claim 1, wherein the rear side of the body has vertical folds.
